(19) United States
(12) Patent Application Publication
(10) Pub. No.: US 2024/0150851 A1
(43) Pub. Date: May 9, 2024

(54) RAPID, NON-INVASIVE DETECTION AND SERIAL MONITORING OF INFECTIONS IN SUBJECTS USING MICROBIAL CELL-FREE DNA SEQUENCING

(71) Applicant: Karius, Inc., Redwood City, CA (US)

(72) Inventors: Matthew Smollin, Redwood City, CA (US); Nick Degner, Redwood City, CA (US); Ozlem Equils, Redwood City, CA (US); Aparna Arun, Redwood City, CA (US); Christiaan Devries, Redwood City, CA (US); Ann MacIntyre, Redwood City, CA (US); Varsha Baichwal, Redwood City, CA (US); Asim Ahmed, Redwood City, CA (US)

(21) Appl. No.: 18/349,071

(22) Filed: Jul. 7, 2023

Related U.S. Application Data
(63) Continuation of application No. PCT/US2022/011867, filed on Jan. 10, 2022.
(60) Provisional application No. 63/136,060, filed on Jan. 11, 2021.
(60) Provisional application No. 63/136,076, filed on Jan. 11, 2021.

(51) Int. Cl. C12Q 1/689; C12Q 1/6874; C12Q 1/6895

(52) U.S. Cl. CPC C12Q 1/689; C12Q 1/6874; C12Q 1/6895

(57) ABSTRACT

This disclosure provides methods for detecting microbial cell-free nucleic acids for use in a wide variety of contexts. In some embodiments, this disclosure provides methods of monitoring a response to treatment. In some embodiments, this disclosure provides methods applicable for detecting bacterial infections (e.g., Bartonella spp. infections) and/or fungal infections. In some embodiments, the microbial cell-free nucleic acids are detected by metagenomic next-generation sequencing.

### CROSS-REFERENCE

This application is a continuation application of International Patent Application No. PCT/US2022/011867, filed Jan. 10, 2022, which claims the benefit of U.S. Provisional Patent Application 63/136,060, filed on Jan. 11, 2021, and U.S. Provisional Patent Application 63/136,076, filed on Jan. 11, 2021, each of which applications are incorporated herein by reference in their entireties.

### BACKGROUND

A diverse spectrum of invasive molds and fungi cause serious opportunistic infections in immunocompromised (IC) children. Their overlap in clinical presentation can make it challenging to differentiate among etiologies and optimally tailor antifungal therapy. Current methods to identify these pathogens generally lack sensitivity, are limited by long turnaround times, and require an array of individual tests on invasively obtained specimens. The delay or lack of a pathogen diagnosis in combination with the reliance on invasive procedures can lead to a dependence on broad empiric therapy, the development of antimicrobial resistance and increases in morbidity and mortality.

There are several hindrances to the diagnosis of Bartonella infections: (1) the fastidious nature of Bartonella spp., leads to rare detections with traditional culture-based methods and a lack of reliable, widely available diagnostic tests; (2) the nonspecific manifestations of the disease; and (3) failure to obtain a history of exposure risk factors for Bartonella infection.

Among other things, the following disclosure offers rapid, non-invasive assays to detect bacterial and fungal infections through next-generation sequencing (NGS) of plasma microbial cell-free DNA (mcfDNA) and can overcome many of these limitations.

### SUMMARY

One aspect of the disclosure herein is a method of monitoring a treatment regimen for a bacterial infection in a patient comprising (a) preparing an initial plasma sample comprising microbial cell-free nucleic acids (mcfNA) from the patient, wherein the initial plasma sample is collected from the patient before beginning the treatment regimen for the bacterial infection or while the patient is undergoing the treatment regimen for the bacterial infection; (b) measuring a threshold amount of bacterial mcfNA in the initial plasma sample (optionally, relative to a standard such as synthetic nucleic acids), wherein the bacterial mcfNA is from bacteria associated with the bacterial infection; (c) preparing a longitudinal plasma sample comprising bacterial mcfNA from the patient, wherein the longitudinal plasma sample is collected from the patient at least a day after the initial plasma sample; (d) measuring a second amount of bacterial mcfNA in the longitudinal plasma sample, wherein the bacterial mcfNA is from bacteria associated with the bacterial infection; and (e) repeating (c) and (d) and maintaining the treatment until the second amount of the bacterial mcfNA in the longitudinal plasma sample is significantly lower than the threshold amount of the bacterial mcfNA. In one embodiment of the methods disclosed herein, the microbial infection is a bacterial infection, preferably Bartonella spp., more preferably Bartonella henselae or Bartonella quintana. In another embodiment, the patient has cat-scratch fever, trench foot, endocarditis, or osteomyelitis. In another embodiment, the patient has a glomerulonephritis or fever. In another embodiment, the microbial infection is localized in a tissue selected from the group consisting of cardiac tissue, mitral valve, cardiac sac, aorta, lung, liver, and cardiac cells. In another embodiment, the microbial infection causes a disease selected from the group consisting of abscess, septic emboli, osteomyelitis, arthritis, psoas abscess, and endocarditis. In another embodiment, the patient has endocarditis, specifically native valve endocarditis or prosthetic valve endocarditis. In some cases, the treatment comprises a prosthetic valve replacement. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

Another aspect of the disclosure herein is a method of treating a bacterial infection in a patient comprising (a) preparing an initial plasma sample comprising mcfNA from the patient; (b) measuring a threshold concentration of mcfNA in the initial plasma sample, wherein the mcfNA is associated with the bacterial infection in the patient; (c) administering a treatment to the patient for the microbial infection; (d) preparing a longitudinal plasma sample comprising mcfNA collected from the patient after (c); (e) measuring a second amount of mcfNA in the longitudinal plasma sample, wherein the mcfNA is associated with the bacterial infection in the patient; (f) treating the patient for the microbial infection if the second amount of mcfNA is substantially greater than the threshold amount of mcfNA; and (g) repeating (c)-(f) until the amount of mcfNA in a longitudinal plasma sample is significantly lower than the threshold amount of mcfNA. In one embodiment of the methods disclosed herein, the microbial infection is a bacterial infection, preferably Bartonella spp., more preferably Bartonella henselae or Bartonella quintana. In another embodiment, the patient has cat-scratch fever, trench foot, endocarditis, or osteomyelitis. In another embodiment, the patient has a glomerulonephritis or fever. In another embodiment, the microbial infection is localized in a tissue selected from the group consisting of cardiac tissue, mitral valve, cardiac sac, aorta, lung, liver, and cardiac cells. In another embodiment, the microbial infection causes a disease selected from the group consisting of abscess, septic emboli, osteomyelitis, arthritis, psoas abscess, and endocarditis. In another embodiment, the patient has endocarditis, specifically native valve endocarditis or prosthetic valve endocarditis. In some cases, the treatment comprises a prosthetic valve replacement. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

Another aspect of the disclosure herein is a method of detecting a bacterial infection in a patient comprising (a) preparing an initial plasma sample comprising mcfNA and a known amount of synthetic spike-in nucleic acids (sNA); (b) analyzing the mcfNA to identify the bacterial infection; (c) measuring a threshold amount of mcfNA in the initial plasma sample relative to the sNA, wherein the bacterial mcfNA is from at least one bacterium associated with the bacterial infection; (d) preparing a longitudinal plasma sample comprising mcfNA and a known amount of a second sNA; (e) measuring a second amount of mcfNA in the longitudinal plasma sample relative to the second sNA, wherein the mcfNA is from at least one bacterium associated with the bacterial infection; and (f) repeating (c)-(e) until the second amount of bacterial mcfNA in a longitudinal plasma sample is significantly lower than the threshold amount of bacterial mcfNA in the initial plasma sample. In one embodiment of the methods disclosed herein, the microbial infection is a bacterial infection, preferably Bartonella spp., more preferably Bartonella henselae or Bartonella quintana. In another embodiment, the patient has cat-scratch fever, trench foot, endocarditis, or osteomyelitis. In another embodiment, the patient has a glomerulonephritis or fever. In another embodiment, the microbial infection is localized in a tissue selected from the group consisting of cardiac tissue, mitral valve, cardiac sac, aorta, lung, liver, and cardiac cells. In another embodiment, the microbial infection causes a disease selected from the group consisting of abscess, septic emboli, osteomyelitis, arthritis, psoas abscess, and endocarditis. In another embodiment, the patient has endocarditis, specifically native valve endocarditis or prosthetic valve endocarditis. In some cases, the treatment comprises a prosthetic valve replacement. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

One aspect of the disclosure herein is a method of monitoring a treatment regime for a fungal infection in a patient comprising (a) preparing an initial plasma sample comprising microbial cell-free nucleic acids (mcfNA) from the patient, wherein the initial plasma sample is collected from the patient before beginning the treatment regimen for the fungal infection or while the patient is undergoing the treatment regimen for the fungal infection; (b) measuring a threshold amount of fungal mcfNA in the initial plasma sample, wherein the fungal mcfNA is from at least one fungus associated with the fungal infection; (c) preparing a longitudinal plasma sample comprising fungal mcfNA from the patient, wherein the longitudinal plasma sample is collected from the patient at least a day after the initial plasma sample; (d) measuring a second amount of the fungal mcfNA in the longitudinal plasma sample, wherein the fungal mcfNA is from at least one fungus associated with the fungal infection; and (e) repeating (c) and (d) and maintaining the treatment until the second amount of the fungal mcfNA in the longitudinal plasma sample is significantly lower than the threshold amount of the fungal mcfNA. In one embodiment of the methods disclosed herein, the microbial infection is a fungal infection. In another embodiment, the fungal infection is caused by a fungus selected from the group consisting of Aspergillus, Pneumocystis, Rhizopus, Cunninghamella, Mucor, Lichtheimia, and Rhizomucor. In another embodiment, the fungus is selected from the group consisting of Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, and Rhizomucor meihei. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

Another aspect of the disclosure herein is a method of treating a fungal infection in a patient comprising (a) preparing an initial plasma sample comprising mcfNA from the patient; (b) measuring a threshold amount of mcfNA in the initial plasma sample, wherein the mcfNA is associated with the fungal infection in the patient; (c) administering a treatment to the patient for the fungal infection; (d) preparing a longitudinal plasma sample comprising mcfNA collected from the patient after (c); (e) measuring a second amount of mcfNA in the longitudinal plasma sample, wherein the mcfNA is associated with the fungal infection in the patient; (f) treating the patient for the microbial infection if the second amount of mcfNA is substantially greater than the threshold amount of mcfNA; and (g) repeating (c)-(f) until the second amount of mcfNA is significantly lower than the threshold amount of mcfNA. In one embodiment of the methods disclosed herein, the microbial infection is a fungal infection. In another embodiment, the fungal infection is caused by a fungus selected from the group consisting of Aspergillus, Pneumocystis, Rhizopus, Cunninghamella, Mucor, Lichtheimia, and Rhizomucor. In another embodiment, the fungus is selected from the group consisting of Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, and Rhizomucor meihei. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

Another aspect of the disclosure herein is a method of detecting a fungal infection in a patient comprising (a) preparing an initial plasma sample comprising microbial cell-free nucleic acids (mcfNA) from the patient and a known amount of synthetic spike-in nucleic acids (sNA); (b) analyzing the mcfNA to identify or detect the fungal infection; (c) measuring a threshold amount of mcfNA in the initial plasma sample relative to the sNA, wherein the mcfNA is from at least one fungus associated with the fungal infection; (d) preparing a longitudinal plasma sample comprising mcfNA and a known amount of a second sNA; (e) measuring a second amount of mcfNA in the longitudinal plasma sample relative to the second sNA, wherein the mcfNA is from at least one fungus associated with the fungal infection; and (f) repeating (c)-(e) until the second amount of mcfNA in the longitudinal plasma sample is significantly lower than the threshold amount of mcfNA in the initial plasma sample. In one embodiment of the methods disclosed herein, the microbial infection is a fungal infection. In another embodiment, the fungal infection is caused by a fungus selected from the group consisting of Aspergillus, Pneumocystis, Rhizopus, Cunninghamella, Mucor, Lichtheimia, and Rhizomucor. In another embodiment, the fungus is selected from the group consisting of Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, and Rhizomucor meihei. In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

In one embodiment of the methods disclosed herein, the microbial infection is a bacterial infection, preferably Bartonella spp., more preferably Bartonella henselae or Bartonella quintana. In another embodiment, the patient has cat-scratch fever, trench foot, endocarditis, or osteomyelitis. In another embodiment, the patient has a glomerulonephritis or fever. In another embodiment, the microbial infection is localized in a tissue selected from the group consisting of cardiac tissue, mitral valve, cardiac sac, aorta, lung, liver, and cardiac cells. In another embodiment, the microbial infection causes a disease selected from the group consisting of abscess, septic emboli, osteomyelitis, arthritis, psoas abscess, and endocarditis. In another embodiment, the patient has endocarditis, specifically native valve endocarditis or prosthetic valve endocarditis. In some cases, the treatment comprises a prosthetic valve replacement.

In one embodiment of the methods disclosed herein, the microbial infection is a fungal infection. In another embodiment, the fungal infection is caused by a fungus selected from the group consisting of Aspergillus, Pneumocystis, Rhizopus, Cunninghamella, Mucor, Lichtheimia, and Rhizomucor. In another embodiment, the fungus is selected from the group consisting of Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, and Rhizomucor meihei.

In one embodiment of the methods disclosed herein, the subject is immunocompromised. In another embodiment, the subject has received an immunosuppressant. In another embodiment, the patient has a negative blood culture. In another embodiment, the patient has a metastatic infection. In another embodiment, the patient is febrile. In another embodiment, the treatment comprises administering a drug selected from the group consisting of a penicillin, tetracycline, cephalosporin, and aminoglycoside. In another embodiment, the treatment comprises administering a drug is selected from the group consisting of vancomycin, cefepime, meropenem, and doxycycline. In one embodiment of the methods disclosed herein, the mcfNA concentration is measured by metagenomic next generation sequencing. In another embodiment, the mcfNA is DNA or RNA. In one embodiment of the methods disclosed herein, the amount of mcfNA in the longitudinal plasma sample is 10%-100% lower than the threshold value, 25%-100% lower than the threshold value, 50%-100% lower than the threshold value or 75%-100% lower than the threshold value. In another embodiment, the methods further comprise detecting herpesvirus in the initial plasma sample and/or detecting herpesvirus in the longitudinal plasma sample.

Another aspect of the disclosure herein is a non-invasive method of detecting the presence and amount of at least one pathogen in a subject at risk for a pulmonary infection comprising: (a) providing a plasma sample comprising cell-free nucleic acids from the subject; (b) determining the sequence of the microbial cell-free nucleic acids in the sample to obtain microbial sequence reads; and (c) using the microbial sequence reads to detecting the presence and amount of at least one pathogen in a sample from the subject. In one embodiment of the method, at least one pathogen is a fungus, preferably a fungus selected from the group consisting of: Aspergillus, Pneumocystis, Rhizopus, Cunninghamella, Mucor, Lichtheimia, and Rhizomucor, more preferably at least one fungus selected from the group consisting of: Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, and Rhizomucor meihei. In another embodiment the subject is immunocompromised, for example, the subject has received an immunosuppressant. In another embodiment, at least one pathogen is identified at the genus, strain, or species level. In yet another embodiment the at least one pathogen is identified at the strain or species level.

Another aspect of the disclosure herein is a non-invasive method of detecting an elevated infection risk in an immunocompromised subject comprising the steps of: (a) providing a plasma sample comprising cell-free nucleic acids from the subject; (b) determining the sequence of the microbial cell-free nucleic acids in the sample to obtain microbial sequence reads; (c) using the microbial sequence reads to detecting the presence and amount of at least one pathogen in a sample from the subject; (d) comparing the amount of at least one pathogen to a predetermined threshold; and (e) detecting an elevated infection risk if the amount of the at least one pathogen exceeds the predetermined threshold. In one embodiment of the method, the immunocompromised subject is an HIV/AIDS patient, preferably a subject who has received an immunosuppressant. In another embodiment, at least one pathogen is a fungus, preferably a fungus from the group comprising Aspergillus, Pneumocystis, Rhizopus, Cunninghamella, Mucor, Lichtheimia, and Rhizomucor. In another embodiment, the presence and amount of at least two pathogens are determined and wherein at least one pathogen is a fungus and at least one pathogen is a virus, bacterium, or parasite. In another embodiment, the pathogen is a virus selected from the group comprising a DNA virus, a herpes virus, and a cytomegalovirus. In another embodiment, the pathogen is identified at the genus, strain, or species level.

Another aspect of the disclosure herein is a non-invasive method of monitoring response to an anti-fungal treatment comprising the steps of (a) providing a first plasma sample from the subject, wherein the sample comprises fungal cell-free nucleic acids; (b) performing high throughput sequencing of the fungal cell-free nucleic acids to obtain fungal cell free nucleic acid reads; (c) using the fungal cell free nucleic acid reads to identify the presence and amount of at least one fungus in the sample; (d) providing a second plasma sample from the subject, wherein the sample comprises fungal cell-free nucleic acids; (e) performing high throughput sequencing of the fungal cell-free nucleic acids to obtain fungal cell free nucleic acid reads; (g) using the fungal cell free nucleic acid reads to identify the presence and amount of at least one fungus in the sample; and (h) comparing the amount of at least one fungus in the first and second samples. In one embodiment of the method, the first plasma sample was obtained from the subject at a first time point and the second plasma sample was obtained from the subject at a second time point. In another embodiment, the method further comprises administering an anti-fungal treatment to the subject.

Another aspect of the disclosure is a method for treating a patient with an anti-fungal treatment, wherein the patient is immunocompromised, the method comprising: (a) detecting an elevated risk of fungal infection in a patient by (i) obtaining or having obtained a plasma sample comprising cell-free nucleic acids from the patient; (ii) determining the sequence of the fungal cell-free nucleic acids in the sample to obtain fungal sequence reads; (iii) using the fungal sequence reads to detecting the presence and amount of at least one fungus in a sample from the patient; (iv) comparing the amount of at least one pathogen to a predetermined threshold; and (v) detecting an elevated risk of fungal infection in the patient, if the amount of the at least one pathogen exceeds the predetermined threshold; and (b) if the patient has an elevated risk of fungal infection, then administering an anti-fungal treatment to the patient.

Another aspect of the disclosure is a non-invasive method of detecting a pathogen in a subject at risk for endocarditis comprising the steps of: (a) obtaining a plasma sample comprising cell-free nucleic acids from the subject; (b) determining the sequence of the microbial cell-free nucleic acids in the sample; and (c) determining the presence and amount of at least one pathogen. In one embodiment of the method, the subject has a prosthetic heart valve. In another aspect, the pathogen is selected from the group of fastidious pathogens consisting of Bartonella henselae and Bartonella quintana. In another embodiment, the subject at risk for endocarditis is exhibiting at least one endocarditis related symptom. In another embodiment, the subject is exhibiting at least one of glomerulonephritis and fever. In another embodiment, the prosthetic heart valve is selected from the group comprising a partial heart valve and a complete heart valve.

Another aspect of the disclosure herein is a method of detecting the presence of a fastidious pathogen in a sample from a subject exhibiting a fever comprising (a) obtaining a plasma sample comprising cell-free nucleic acids from the subject; (b) determining the sequence of the microbial cell-free nucleic acids in the sample; and (c) determining the presence and amount of at least one fastidious pathogen. In one embodiment of the method, the subject is at risk for a condition selected from the group comprising cat-scratch fever, trench foot, endocarditis, osteomyelitis. In another embodiment, the fastidious pathogen is selected from the group consisting of: Bartonella henselae and Bartonella quintana.

Another aspect of the disclosure herein is a method of monitoring a response to an antibacterial treatment in a subject at comprising the steps of (a) providing a first plasma sample from the subject, wherein the sample comprises bacterial cell-free nucleic acids; (b) performing high throughput sequencing of the bacterial cell-free nucleic acids to obtain bacterial cell free nucleic acid reads; (c) using the bacterial cell free nucleic acid reads to identify the presence and amount of at least one bacterium in the sample; (d) providing a second plasma sample from the subject, wherein the sample comprises bacterial cell-free nucleic acids; (e) performing high throughput sequencing of the bacterial cell-free nucleic acids to obtain bacterial cell free nucleic acid reads; (g) using the bacterial cell free nucleic acid reads to identify the presence and amount of at least one bacterium in the sample; and (h) comparing the amount of at least bacterium in the first and second samples. In one embodiment of the method, the bacterium is Bartonella henselae or Bartonella quintana. In another embodiment, the method further comprises administering an antibacterial treatment to the subject between the steps of providing a first sample from the subject and providing a second sample from the subject.

Another aspect of the disclosure herein is a method of monitoring the risk of infection in a subject who has received a prosthetic valve replacement comprising the steps of (a) providing a first plasma sample from the subject; (b) performing high throughput sequencing of the cell-free nucleic acids to obtain cell free nucleic acid reads; (c) using the cell free nucleic acid reads to identify the presence and amount of at least one pathogen in the sample; (d) comparing the amount of at least one pathogen to a threshold level and (e) determining the subject is at risk for infection if the amount of the pathogen exceeds a threshold level. In one embodiment of the method, the pathogen is a bacterium, preferably Bartonella henselae or Bartonella quintana. In another embodiment, the prosthetic heart valve is selected from the group comprising a partial heart valve and a complete heart valve.

Another aspect of the disclosure herein is a non-invasive method of detecting a bacterial infection at a site of localization in a subject with a fever, comprising a) obtaining a plasma sample from the subject, (b) determining the sequence of microbial cell-free nucleic acids (e.g., bacterial cell-free nucleic acids) in the sample; (c) comparing the amount of microbial cell-free nucleic acids (e.g., bacterial cell-free nucleic acids)acids to a threshold level; (d) determining the amount of cell free nucleic acids from at least one bacterium; (e) detecting a bacterial infection if the amount of microbial cell-free nucleic acids (e.g., bacterial cell-free nucleic acids) exceeds a threshold level. In one embodiment of the method, the site of localization is selected from the group comprising the heart, mitral valve, lung, liver, kidney, cardiac tissue, cardiac sac, aorta, and cardiac cells. In another embodiment, the method further comprises administering a treatment regimen. In another embodiment, the bacterium is Bartonella henselae or Bartonella quintana.

One aspect of the disclosure herein is a method of monitoring a treatment regime for a bacterial infection in a patient comprising (a) preparing an initial sample comprising microbial cell-free nucleic acids (mcfNA) from the patient, wherein the initial sample is collected from the patient before beginning the treatment regimen for the bacterial infection or while the patient is undergoing the treatment regimen for the bacterial infection and wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) measuring a threshold amount of bacterial mcfNA in the initial sample (optionally, relative to a standard such as synthetic nucleic acids), wherein the bacterial mcfNA is from bacteria associated with the bacterial infection; (c) preparing a longitudinal sample comprising bacterial mcfNA from the patient, wherein the longitudinal sample is collected from the patient at least a day after the initial sample; (d) measuring a second amount of bacterial mcfNA in the longitudinal sample, wherein the bacterial mcfNA is from bacteria associated with the bacterial infection; and (e) repeating (c) and (d) and maintaining the treatment until the second amount of the bacterial mcfNA in the longitudinal sample is significantly lower than the threshold amount of the bacterial mcfNA.

Another aspect of the disclosure herein is a method of treating a bacterial infection in a patient comprising (a) preparing an initial sample comprising mcfNA from the patient, wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) measuring a threshold concentration of mcfNA in the initial sample, wherein the mcfNA is associated with the bacterial infection in the patient; (c) administering a treatment to the patient for the microbial infection; (d) preparing a longitudinal sample comprising mcfNA collected from the patient after (c); (e) measuring a second amount of mcfNA in the longitudinal sample, wherein the mcfNA is associated with the bacterial infection in the patient; (f) treating the patient for the microbial infection if the second amount of mcfNA is substantially greater than the threshold amount of mcfNA; and (g) repeating (c)-(f) until the amount of mcfNA in a longitudinal sample is significantly lower than the threshold amount of mcfNA.

Another aspect of the disclosure herein is a method of detecting a bacterial infection in a patient comprising (a) preparing an initial sample comprising mcfNA and a known amount of synthetic spike-in nucleic acids (sNA), wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) analyzing the mcfNA to identify the bacterial infection; (c) measuring a threshold amount of mcfNA in the initial sample relative to the sNA, wherein the bacterial mcfNA is from at least one bacterium associated with the bacterial infection; (d) preparing a longitudinal sample comprising mcfNA and a known amount of a second sNA; (e) measuring a second amount of mcfNA in the longitudinal sample relative to the second sNA, wherein the mcfNA is from at least one bacterium associated with the bacterial infection; and (f) repeating (c)-(e) until the second amount of bacterial mcfNA in a longitudinal sample is significantly lower than the threshold amount of bacterial mcfNA in the initial sample.

One aspect of the disclosure herein is a method of monitoring a treatment regime for a fungal infection in a patient comprising (a) preparing an initial sample comprising microbial cell-free nucleic acids (mcfNA) from the patient, wherein the initial sample is collected from the patient before beginning the treatment regimen for the fungal infection or while the patient is undergoing the treatment regimen for the fungal infection, and wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) measuring a threshold amount of fungal mcfNA in the initial sample, wherein the fungal mcfNA is from at least one fungus associated with the fungal infection; (c) preparing a longitudinal sample comprising fungal mcfNA from the patient, wherein the longitudinal sample is collected from the patient at least a day after the initial sample; (d) measuring a second amount of the fungal mcfNA in the longitudinal sample, wherein the fungal mcfNA is from at least one fungus associated with the fungal infection; and (e) repeating (c) and (d) and maintaining the treatment until the second amount of the fungal mcfNA in the longitudinal sample is significantly lower than the threshold amount of the fungal mcfNA.

Another aspect of the disclosure herein is a method of treating a fungal infection in a patient comprising (a) preparing an initial sample comprising mcfNA from the patient wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) measuring a threshold amount of mcfNA in the initial sample, wherein the mcfNA is associated with the fungal infection in the patient; (c) administering a treatment to the patient for the fungal infection; (d) preparing a longitudinal sample comprising mcfNA collected from the patient after (c); (e) measuring a second amount of mcfNA in the longitudinal sample, wherein the mcfNA is associated with the fungal infection in the patient; (f) treating the patient for the microbial infection if the second amount of mcfNA is substantially greater than the threshold amount of mcfNA; and (g) repeating (c)-(f) until the second amount of mcfNA is significantly lower than the threshold amount of mcfNA.

Another aspect of the disclosure herein is a method of detecting a fungal infection in a patient comprising (a) preparing an initial sample comprising microbial cell-free nucleic acids (mcfNA) from the patient and a known amount of synthetic spike-in nucleic acids (sNA), wherein the sample is a cell-free sample, a bodily-fluid sample, or a sample selected from the group consisting of blood, plasma, serum, urine, stool, saliva, lymph, spinal fluid, synovial fluid, and bronchoalveolar lavage; (b) analyzing the mcfNA to identify or detect the fungal infection; (c) measuring a threshold amount of mcfNA in the initial sample relative to the sNA, wherein the mcfNA is from at least one fungus associated with the fungal infection; (d) preparing a longitudinal sample comprising mcfNA and a known amount of a second sNA; (e) measuring a second amount of mcfNA in the longitudinal sample relative to the second sNA, wherein the mcfNA is from at least one fungus associated with the fungal infection; and (f) repeating (c)-(e) until the second amount of mcfNA in the longitudinal sample is significantly lower than the threshold amount of mcfNA in the initial sample.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the results of serial test sampling of a Bartonella infection. Molecules per microliter (MPM) is measured over several days after a threshold test. Data was collected from three patients: (●) one with native valve endocarditis; (▪) one with prosthetic valve endocarditis; and (▴) one with fever of unknown origin.

FIG. 2 shows the results of serial test sampling of a fungal infection. Molecules per microliter (MPM) is measured over several days after a threshold test. FIG. 2A shows results from patients infected with non-Aspergillus sp. molds. The following codes are used to identify individuals: R-O-1_BMT-L; R-MIC-2_BMT-P; R-O-2_UK-UK; R-O-3_HM-OG; R-O-4_SOT-UK; R-O-5_HM-IA; C-1_HM-P; C-2_AA-P; M-I-1_BMT-SY; M-I-2_BMT-LS; L-C-1_HM-P; R-ME-1_UK-P*; R-P-1 AA-P. FIG. 2B shows results from patients infected with Aspergillus. The following codes are used to identify individuals: AF-1_HM-P; AF-2_ST-P; AF-3_BMT-P; AF-4_SOT-P; AF-5_ISM-P; AF-7_NA-P*; AF-11_UK-P*; AF-12_NA-SY; AF-13_SOT-P; AF-14_CC-P; AF-15_AA-P; A-c-2_AA-P; A-fl-1_HM-P; A-fl-2_UK-UK; A-fl-3_BMT-P*; A-fl-4_HM-H. FIG. 2C shows results from patients infected with PJP. The following codes are used to identify individuals: PJ-1_HM-P; PJ-2_HM-P; PJ-9_BMT-P, PJ-10_BMT-P. FIG. 2D shows a composite of FIG. 2A, FIG. 2B, and FIG. 2C The following coding was used in FIG. 2A, FIG. 2B, FIG. 2C and FIG. 2D: Genus/Species: AF—Aspergillus fumigatus; A-c—Aspergillus calidoustus; A-fl—Aspergillus flavus oryzae; R-D—Rhizopus delemar; R-MIC—Rhizopus microsporus; R-O—Rhizopus oryzae; C—Cunninghamella; M-I—Mucor indicus; L-C—Lichtheimia corymbifera; R-MIE—Rhizomucor miehei; R-P—Rhizopus pusillus; PJ—Pneumocystis jirovecii. Underlying conditions: HM—Hematologic Malignancy; SOT—Solid Organ Transplant; BMT—Bone Marrow Transplant; ST—Solid Tumor; ISM—Immunosuppressing Medications; CC—Cardiac Congenital Disease; AA—Aplastic Anemia; UK—Unknown; NA—None. Focus on indications: P—Pulmonary; SY—Systemic; H—Heart; LS—Liver/Spleen; IA—Intra-abdominal; UK—Unknown.

### DETAILED DESCRIPTION

Disclosed herein in some embodiments, are methods for detecting microbial cell-free nucleic acids (e.g., microbial cell-free DNA “mcfDNA”) in a subject (e.g., patient) in order to detect or monitor the subject's response to an antimicrobial treatment (e.g., antibiotic, antifungal, or antibiotic). In some instances, the subject is being treated for an infection such as a localized infection. For example, the localized infection can be endocarditis, particularly blood culture-negative endocarditis. In some cases, the endocarditis is native valve endocarditis. In some cases, the endocarditis is prosthetic valve endocarditis. In some embodiments, the infection is a Bartonella infection. In some cases, the infection is a fungal infection such as Aspergillus or non-Aspergillus mold. In some cases, the infection is a pulmonary infection such as pneumonia. In some cases, the infection is localized to an organ. In some cases, the infection is localized to the heart, mitral valve, lung, liver, kidney, cardiac tissue, cardiac sac, and/or aorta. The methods provided herein are particularly useful for fastidious or unculturable microbes (e.g., pathogens). Generally, the methods provided herein involve detection and/or quantification of microbial cell free nucleic acids (e.g., microbial cell-free DNA, microbial cell-free RNA) in a sample from a subject (e.g., plasma). In some cases, this disclosure provides methods method of monitoring a treatment of a microbial infection in a patient comprising (a) preparing an initial sample (e.g., plasma) comprising microbial cell-free nucleic acids (mcfNA) from the patient; (b) measuring a threshold amount of mcfNA in the initial plasma sample; (c) preparing a longitudinal sample (e.g., plasma sample) comprising mcfNA and a known amount of a second sNA; (d) measuring a second mcfNA concentration in the longitudinal plasma sample relative to the second sNA; and (e) repeating (c) and (d) and maintaining the treatment until the mcfNA concentration in the longitudinal blood sample is significantly lower than the threshold mcfNA concentration. In some cases, this disclosure provides a method of treating a microbial infection in a patient comprising (a) preparing an initial plasma sample comprising microbial cell-free nucleic acids (mcfNA); (b) measuring a threshold concentration of mcfNA in the initial plasma sample; (c) treating the patient for the microbial infection; (d) preparing a longitudinal plasma sample comprising mcfNA; (e) measuring a second mcfNA concentration in the longitudinal plasma sample; (f) treating the patient for the microbial infection when the second mcfNA concentration is substantially greater than the threshold mcfNA concentration; and (g) repeating (c)-(f) until the mcfNA concentration in a longitudinal blood sample is significantly lower than the threshold mcfNA concentration. In some cases, the method is a method of detecting a microbial infection in a patient comprising (a) preparing an initial plasma sample comprising microbial cell-free nucleic acids (mcfNA); (b) analyzing mcfNA to identify the microbial infection at a species or strain level; (c) measuring a threshold concentration of mcfNA in the initial plasma sample relative to the sNA; (d) preparing a longitudinal plasma sample comprising the mcfNA and a known amount of a second sNA; (e) measuring a second mcfNA concentration in the longitudinal plasma sample relative to the second sNA; and (f) repeating (d) and (e) until the mcfNA concentration in a longitudinal blood sample is significantly lower than the threshold mcfNA concentration.

In some cases, the sample (e.g., plasma sample) is spiked with a known concentration of synthetic DNA for quality control purposes. The methods can comprise attaching a nucleic acid adapter (e.g., DNA adapter) to the cell-free nucleic acids (e.g., cell-free DNA) and preparing a sequencing library. In some cases, the methods comprise attaching a first adapter to DNA from a first subject and a second adapter comprising a different sequence to a DNA sample from a second subject to produce first and second DNA libraries respectively. In some cases, the first and second DNA libraries are combined. The libraries may be subjected to multiplex sequencing (e.g., next generation sequencing, metagenomic sequencing), after which the sequence reads are demultiplexed. In some cases, samples (or libraries derived therefrom) from multiple subjects (e.g., at least 2, 3, 4, 5, 7, 10 subjects) are combined during the process of multiplex sequencing. In some cases, the sequencing comprises performing sequencing-by-synthesis reactions using reversible terminators, particularly fluorescently labeled reversible terminators (e.g., fluorescently labeled ddNTP, dNTP). In some embodiments, sequence reads exhibiting strong alignment against human references or the synthetic molecule references are excluded from the analysis. In some cases, sequence reads are filtered based on sequencing quality. In some embodiments, the remaining reads are aligned against a microorganism database. In some embodiments, an expectation maximization algorithm is applied to compute the maximum likelihood estimate of each taxon abundance. In some cases, quantity of each microbe is expressed as Molecules per Microliter (MPM), which can refer to the number of DNA sequence reads from the reported microbe (e.g., bacterium, fungus, virus) present per microliter of plasma, or other biological fluid. In some embodiments, the method further comprises treating the subject for the infection, such as by administering a treatment, maintaining a treatment, or adjusting a dose of treatment. In some cases, the treatment is an antimicrobial treatment (e.g., antibiotic, or antifungal drug). In some cases, the treatment is a broad-spectrum drug. In some cases, the treatment specifically targets a particular microbe.

The methods provided herein generally have the advantage of being rapid and non-invasive. In some cases, the process from DNA extraction to analysis is completed in at most 20 hours, at most 24 hours, at most 28 hours, at most 30 hours, at most 36 hours, or at most 48 hours.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

Numeric ranges are inclusive of the numbers defining the range. The term “about” as used herein generally means plus or minus ten percent (10%) of a value, inclusive of the value, unless otherwise indicated by the context of the usage. For example, “about 100” refers to any number from 90 to 110.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

The term “attach” and its grammatical equivalents may refer to connecting two molecules using any mode of attachment. For example, attaching may refer to connecting two molecules by chemical bonds or other method to generate a new molecule. Attaching an adapter to a nucleic acid may refer to forming a chemical bond between the adapter and the nucleic acid. In some cases, attaching is performed by ligation, e.g., using a ligase. For example, a nucleic acid adapter may be attached to a target nucleic acid by ligation, via forming a phosphodiester bond catalyzed by a ligase. In some cases, an adapter can be attached to a target nucleic acid (or copy thereof) using a primer extension reaction.

As used herein, the term “or” is used to refer to a nonexclusive or, such as “A or B” includes “A but not B,” “B but not A,” and “A and B,” unless otherwise indicated.

As used herein, “a”, “an”, and “the” can include plural referents unless otherwise limited expressly or by context.

Subjects

The term “subject” as used herein includes patients, particularly human patients. The term “subject” also encompasses other mammals, laboratory animals, veterinary animals, dogs, cats, and rodents.

In some embodiments disclosed herein, a subject is at risk of having an infection (e.g., high risk of having an infection), particularly at the time of collecting a sample from the subject. As used herein, a subject with a “high risk” of experiencing an infection is a subject with a risk that is higher than that of a healthy subject. For example, a patient who is immunocompromised is generally at high risk of experiencing an infection when compared to a healthy patient who is not immunocompromised.

In some embodiments, a subject has an infection, particularly at the time of collection of a sample from the subject. In some cases, the subject is at risk of developing in the future one or more symptoms of infection. In some embodiments, the subject has no sign of an infection. In some embodiments, the subject is blood-culture negative at the time of collection of a sample. In some embodiments, the subject is blood-culture positive at the time of collection of a sample. In some cases, culture of a tissue of the subject, e.g., a biopsy tissue or a bodily fluid (e.g., blood) is negative at the time of collection of the sample. In some embodiments, the subject is blood-culture positive at the time of collection of a sample for one or more pathogens and blood culture negative for one or more pathogens that later develop into an infection. In some cases, the subject is blood culture negative for a microbe or pathogen detected or predicted by the methods provided herein at the time of collection of the sample. In some embodiments, a subject has symptoms of infection at the time of collection of a sample or samples from the subject. In some embodiments, a symptom of an infection includes a fever, chills, elevated temperature, fatigue, a cough, congestion, fever, elevated heart rate, low blood pressure, hyperventilation, a sore throat, or any combination thereof. In some embodiments, a fever is a rectal, ear or temporal artery temperature of 100.4° F. (38° C.) or higher, an oral temperature of 100° F. (37.8° C.) or higher, an armpit temperature of 99° F. (37.2° C.) or higher, or any combination thereof.

In some embodiments, the subject is a child. In some embodiments, a child is less than about 18 years of age. In some embodiments, the subject is a pediatric patient. In some embodiments, a subject is an adult. In some embodiments, a subject is less than about 25 years of age. In some embodiments, a subject is elderly. In some embodiments a subject is more than 65 years of age. In some cases, the subject has a high risk of experiencing a bacterial or fungal infection.

In some embodiments, the subject has, is suspected of having, or is at risk (e.g., high risk) of having an infection by a bacterium, a fungus, a virus, a parasite, or any combination thereof, or symptoms of such infection. In some embodiments, the infection is a fungal infection (e.g., invasive fungal infection)). In some embodiments, the infection is a bacterial infection (e.g., localized infection). In some embodiments, a bacterial or fungal infection can comprise an infection by a Bartonella spp bacterium (e.g., Bartonella henselae, Bartonella quintana). In some cases, the microbe is at least one fungus such as Aspergillus, Pneumocystis, Rhizopus, Cunninghamella, Mucor, Lichtheimia, or Rhizomucor. In some cases, the fungus is Aspergillus fumigatus, Aspergillus collidoustus, Aspergillus flavus, Aspergillus oryzae, Pneumocystis jirovecii, Rhizopus delomor, Rhizopus microsporus, Rhizopus oryzae, Rhizopus pusillus, Mucor indicus, Lichtheimia corymbifera, or Rhizomucor meihei. In some cases, the microbe is a herpesvirus, e.g., a reactivating herpesvirus. In some embodiments the microbe or organism is at least one microbe or organism mentioned in the Examples section of this application. In some embodiments, the bacterial infection is a gram-negative bacterial infection. In some embodiments, the bacterial infection is a gram-positive bacterial infection. In some embodiments, the bacterial or fungal infection is susceptible to empirical antimicrobial therapy. In some embodiments, the subject is diagnosed with having an infection or predicted to be at risk of an infection using methods disclosed herein. In some embodiments, a subject is predicted to be at risk of having an infection or at risk of developing symptoms of infection using methods disclosed herein.

A subject can be healthy; or, in some embodiments, the subject has a disease (e.g., cancer, infection) or disorder. In some embodiments, the subject has cancer. In some embodiments, the cancer is a relapsed or refractory cancer. In some embodiments, the cancer is a blood cancer (e.g., leukemia, chronic leukemia, acute leukemia). In some embodiments, the subject is immunocompromised. In some cases, the subject is an immunocompromised child. In some embodiments, the subject has pneumonia. In some embodiments, the pneumonia is Pneumocystis jiroveci pneumonia (PJP).

In some embodiments, the subject is receiving chemotherapy, targeted therapy, immunotherapy, or a combination thereof. In some embodiments, a chemotherapy can comprise an alkylating agent, an antimetabolite, an anti-tumor antibiotic, a topoisomerase inhibitor, a mitotic inhibitor, or any combination thereof. In some embodiments, a subject can be immunosuppressed, e.g., because of the chemotherapy. In some embodiments, a subject is a recipient of a hematopoietic stem cell transplant. In some embodiments, the subject has neutropenia. In some embodiments, the subject does not have neutropenia.

Samples

In some embodiments, a sample is collected from a subject (e.g., a patient). In some embodiments, the sample is a biological sample. The samples analyzed in the methods provided herein are preferably any type of clinical sample. In some cases, the samples contain cells, tissue, or a bodily fluid. In preferred embodiments, the sample is a liquid or fluid sample. In some cases, the sample is a bodily fluid. In some cases, the sample is whole blood, plasma, serum, urine, stool, saliva, lymph, spinal fluid, synovial fluid, bronchoalveolar lavage, nasal swab, respiratory secretions, vaginal fluid, amniotic fluid, semen, or menses. In some cases, the sample is made up of, in whole or in part, cells or tissue. In some cases, cells, cell fragments, or exosomes are removed from the sample, such as by centrifugation or filtration.

In some embodiments, a biological sample is a whole blood sample. In some embodiments, the sample is a cell-free sample, such as a plasma sample or a cell-free plasma sample. In some embodiments, the sample is a sample of isolated or extracted nucleic acids (e.g., DNA, RNA, cell-free DNA). In some embodiments, the plasma sample is collected by collecting blood through venipuncture. In some embodiments, a specimen is mixed with an additive immediately after collection. In some cases, the additive is an anti-coagulant. In some cases, the additive prevents degradation of nucleic acids. In some cases, the additive is EDTA. In some embodiments, measures can be taken to avoid hemolysis or lipemia. In some embodiments, a sample is processed or unprocessed. In some embodiments, a sample is processed by extracting nucleic acids from a biological sample. In some embodiments, DNA is extracted from a sample. In some embodiments, nucleic acids are not extracted from the sample. In some embodiments, a sample comprises nucleic acids. In some embodiments, a sample consists essentially of nucleic acids.

In some cases, the methods provided herein comprise processing whole blood into a plasma sample. In some embodiments, such processing comprises centrifuging the whole blood in order to separate the plasma from blood cells. In some cases, the method further comprises subjecting the plasma to a second centrifugation, often at a higher speed in order to remove bacterial cells and cellular debris. In some cases, the second centrifugation is at a relative centrifugal force (rcf) of least about 4,000 rcf, at least about 5,000 rcf, at least about 6,000 rcf, at least about 8,000 rcf, at least about 10,000 rcf, at least about 12,000 rcf, at least about 14,000 rcf, at least about 16,000 rcf, or at least about 20,000 rcf.

In some cases, the method comprises collecting, obtaining, or providing a sample. In some cases, the method comprises collecting, obtaining, or providing multiple samples, e.g., multiple samples from the subject or patient. In some embodiments, the sample is collected when the subject has an infection. In some cases, the sample is collected prior the subject having an infection. In some cases, the sample is collected while the subject is receiving treatment for an infection. In some cases, the sample is collected after the subject has received a treatment for an infection. In some cases, additional samples are collected from the subject over time. In some embodiments, a second sample is collected from the subject at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 35 days, about 40 days, about 45 days, about 50 days, about 55 days, about 60 days, about 65 days, about 70 days, about 75 days, about 80 days, about 85 days, about 90 days, about 95 days, or about 100 days after the collection of an initial (or other) sample from the subject

In some embodiments, a plurality of samples is collected over a series of time points. In some embodiments, a plurality of samples is collected to monitor an onset of a disease, to monitor progression of a disease, to detect a response to treatment for the disease or any combination thereof. In some embodiments, the plurality of samples is at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected before onset of a symptom. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected over a period of time. In some embodiments, a plurality of samples is collected on consecutive days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples are collected on consecutive days. In some embodiments, a plurality of samples is collected on alternate days. In some embodiments, at least 2 samples, at least 3 samples, at least 4 samples, at least 5 samples, at least 6 samples, at least 7 samples, at least 8 samples, at least 9 samples, at least 10 samples, at least 11 samples, at least 12 samples, at least 13 samples, at least 14 samples, at least 15 samples, at least 16 samples, at least 17 samples, at least 18 samples, at least 19 samples, at least 20 samples, at least 25 samples, at least 30 samples, or at least 35 samples can be collected on alternate days. In some embodiments, the collection of samples can be interspersed between days when no sample is collected. In some embodiments, a schedule of sample collection can repeat over several days. In some embodiments, a schedule of sample collection can repeat over 2 days, over 3 days, over 4 days, over 5 days, over 6 days, over 7 days, over 8 days, over 9 days, over 10 days, over 11 days, over 12 days, over 13 days, over 14 days, over 15 days, over 16 days, over 17 days, over 18 days, over 19 days, over 20 days, over 21 days, or over 22 days. In some embodiments, a schedule of sample collection can repeat on the same day, collecting multiple samples from a subject throughout the 24 hours.

Often, a sample disclosed herein comprises a target nucleic acid (e.g., target DNA, target RNA). In some embodiments, a target nucleic acid is a cell-free nucleic acid. For example, the sample can comprise microbial cell-free nucleic acids (e.g., mcfDNA) that comprises a microbial target DNA (e.g., mcfDNA derived from a microbe, which can include pathogenic microbes). Exemplary microbes that can be detected by the methods provided herein include bacteria, fungi, parasites, and viruses. In some embodiments, a cell-free nucleic acid is a circulating cell-free nucleic acid. In some embodiments, a cell free nucleic acid can comprise cell-free DNA.

In some embodiments, nucleic acids (e.g., cell-free nucleic acids) are extracted from a sample. In some embodiments, isolated nucleic acids (e.g., extracted DNA, extracted RNA) can be used to prepare DNA libraries. In some embodiments, DNA libraries can be prepared by attaching adapters to nucleic acids. In some embodiments, adapters can be used for sequencing of nucleic acids. In some embodiments, nucleic acids can comprise DNA. In some embodiments, nucleic acids containing adapters can be sequenced to obtain sequence reads. In some embodiments, a sample (e.g., a plasma sample comprising mcfDNA) is mixed with adapters prior to extracting nucleic acids or DNA from the sample. In some embodiments, nucleic acids extracted from a sample (e.g., a plasma sample comprising mcfDNA) are attached to adapters following extraction. In some embodiments, sequence reads can be produced through high-throughput sequencing (HTS). In some embodiments, HTS can comprise next-generation sequencing (NGS). In some embodiments, sequence reads can be aligned to sequences in a reference dataset. In some embodiments, sequences can be a bacterial sequence aligned to a reference dataset to obtain an aligned sequence read. In some embodiments, a sequence can be a fungal sequence aligned to a reference dataset to obtain an aligned sequence read. In some embodiments, an aligned bacterial sequence, a fungal sequence, or a combination thereof, can be quantified for bacterial sequences or fungal sequences based on aligned sequence reads obtained.

In the methods provided herein, nucleic acids can be isolated. In some embodiments, nucleic acids can be extracted using a liquid extraction. In some embodiments, a liquid extraction can comprise a phenol-chloroform extraction. In some embodiments, a phenol-chloroform extraction can comprise use of TRIZOL™, DNAZOL™, or any combination thereof. In some embodiments, nucleic acids can be extracted using centrifugation through selective filters in a column. In some embodiments, nucleic acids can be concentrated or precipitated by known methods, including, by way of example only, centrifugation. In some embodiments, nucleic acids can be bound to a selective membrane (e.g., silica) for the purposes of purification. In some embodiments, nucleic acids can be extracted using commercially available kits (e.g., QIAamp CIRCULATING NUCLEIC ACID KIT™, Qiagen DNeasy KIT™, QIAamp KIT™, Qiagen Midi KIT™, QIAprep SPIN KIT™, or any combination thereof). Nucleic acids can also be enriched for fragments of a desired length, e.g., fragments which are less than 1000, 500, 400, 300, 200 or 100 base pairs in length. In some embodiments, enrichment based on size can be performed using, e.g., PEG-induced precipitation, an electrophoretic gel or chromatography material (Huber et al. (1993) Nucleic Acids Res. 21:1061-6), gel filtration chromatography, or TSK gel (Kato et al. (1984) J. Biochem, 95:83-86), which publications are hereby incorporated by reference in their entireties for all purposes.

In some embodiments, a nucleic acid sample can be enriched for a target nucleic acid. In some embodiments, a target nucleic acid is a microbial cell-free nucleic acid.

In some embodiments, target (e.g., pathogen, microbial) nucleic acids are enriched relative to background (e.g., subject) nucleic acids in a sample, for example, by electrophoresis, gel electrophoresis, pull-down (e.g., preferentially pulling down target nucleic acids in a pull-down assay by hybridizing them to complementary oligonucleotides conjugated to a label such as a biotin tag and using, for example, avidin or streptavidin attached to a solid support), targeted PCR, or other methods. Examples of enrichment techniques include but are not limited to: (a) self-hybridization techniques in which a major population in a sample of nucleic acids self-hybridizes more rapidly than a minor population in a sample; (b) depletion of nucleosome-associated DNA from free DNA; (c) removing and/or isolating DNA of specific length intervals; (d) exosome depletion or enrichment; and (e) strategic capture of regions of interest.

In some embodiments, an enriching step can comprise preferentially removing nucleic acids from a sample that are above about 120, about 150, about 200, or about 250 bases in length. In some embodiments, an enriching step comprises preferentially enriching nucleic acids from a sample that are between about 10 bases and about 60 bases in length, between about 10 bases and about 120 bases in length, between about 10 bases and about 150 bases in length, between about 10 bases and about 300 bases in length between about 30 bases and about 60 bases in length, between about 30 bases and about 120 bases in length, between about 30 bases and about 150 bases in length, between about 30 bases and about 200 bases in length, or between about 30 bases and about 300 bases in length. In some embodiments, an enriching step comprises preferentially digesting nucleic acids derived from the host (e.g., subject). In some embodiments, an enriching step comprises preferentially replicating the non-host nucleic acids.

In some embodiments, a nucleic acid library is prepared. In some embodiments, a double-stranded DNA library, a single-stranded DNA library or an RNA library is prepared. A method of preparing a dsDNA library can comprise ligating an adaptor sequence onto one or both ends of a dsDNA fragment. In some cases, the adaptor sequence comprises a primer docking sequence. In some cases, the method further comprises hybridizing a primer to the primer docking sequence and initiating amplification or sequencing of the nucleic acid attached to the adaptor. In some embodiments, the primer or the primer docking sequence comprises at least a portion of an adaptor sequence that couples to a next-generation sequencing platform. In some embodiments, a method can further comprise extension of a hybridized primer to create a duplex, wherein a duplex comprises an original ssDNA fragment and an extended primer strand. In some embodiments, an extended primer strand can be separated from an original ssDNA fragment. In some embodiments, an extended primer strand can be collected, wherein an extended primer strand is a member of an ssDNA library.

In some cases, the library is prepared in an unbiased manner. For example, in some cases, the library is prepared without using a primer that specifically hybridizes to a microbial nucleic acid based on a predetermined sequence of the microbe. For example, in some embodiments, the only amplification performed on the sample involves the use of a primer specific for a sequence of one or more adapters attached to nucleic acids within the sample. In some cases, whole genome amplification is used to prepare the library prior to attachment of the adapters. In some cases, whole genome amplification is not used to prepare the library. In some cases, one or more primers that specifically hybridize to a microbial nucleic acid (e.g., pathogen, viral, fungal, bacterial or parasite nucleic acid) are used to amplify the sample.

In some cases, multiple DNA libraries from different samples (e.g., samples from different patients or subjects) are combined and then subjected to a next generation sequencing assay. In some cases, the libraries are indexed prior to combining in order to track which library corresponds to which sample. Indexing can involve the inclusion of a specific code or bar code in an adapter, e.g., an adapter that is attached to the nucleic acids are to be analyzed. In some cases, the samples comprise a negative control sample or a positive control sample, or both a negative control sample and a positive control sample.

In some embodiments, a length of a nucleic acid can vary. In some embodiments, a nucleic acid or nucleic acid fragment (e.g., dsDNA fragment, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. In some embodiments, a DNA fragment can be about 40 to about 100 bp, about 50 to about 125 bp, about 100 to about 200 bp, about 150 to about 400 bp, about 300 to about 500 bp, about 100 to about 500 bp, about 400 to about 700 bp, about 500 to about 800 bp, about 700 to about 900 bp, about 800 to about 1000 bp, or about 100 to about 1000 bp. In some embodiments, a nucleic acid or nucleic acid fragment (e.g., dsDNA fragment, RNA, or randomly sized cDNA) can be within a range from about 20 to about 200 bp, such as within a range from about 40 to about 100 bp.

In some embodiments, an end of a dsDNA fragment can be polished (e.g., blunt-ended) or be subject to end-repair to create a blunt end. In some embodiments, an end of a DNA fragment can be polished by treatment with a polymerase. In some embodiments, a polishing can involve removal of a 3′ overhang, a fill-in of a 5′ overhang, or a combination thereof. In some embodiments, a polymerase can be a proof-reading polymerase (e.g., comprising 3′ to 5′ exonuclease activity). In some embodiments, a proofreading polymerase can be, e.g., a T4 DNA polymerase, Pol 1 Klenow fragment, or Pfu polymerase. In some embodiments, a polishing can comprise removal of damaged nucleotides (e.g., abasic sites).

In some embodiments, a ligation of an adaptor to a 3′ end of a nucleic acid fragment can comprise formation of a bond between a 3′ OH group of the fragment and a 5′ phosphate of the adaptor. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all 5′ phosphate groups are removed from nucleic acid fragments. In some embodiments, substantially all 5′ phosphates are removed from at least 50%, 55%, 60%, 65%, 70%, 75%, 80%, 85%, 90%, 95%, or greater than 95% of nucleic acid fragments in a sample. Removal of 5′ phosphate groups from a nucleic acid sample can be by any means known in the art. Removal of phosphate groups can comprise treating the sample with heat-labile phosphatase. In some embodiments, 5′ phosphate groups are not removed from the nucleic acid sample. In some embodiments, ligation of an adaptor to the 5′ end of the nucleic acid fragment is performed.

Exemplary Sample Processing

What follows are non-limiting examples of methods provided by this disclosure. In some cases, plasma is spiked with a known concentration of synthetic normalization molecule controls. In some cases, the plasma is then subjected to cell-free NA (cfNA) extraction (e.g., extraction of cell-free DNA). The extracted cfNA can be processed by end-repair and ligated to adapters containing specific indexes to end-repaired cfDNA. The products of the ligation can be purified by beads. In some embodiments, the cfDNA ligated to adapters can be amplified with P5 and P7 primers, and the amplified, adapted cfDNA is purified.

Purified cfDNA attached to adapters derived from a plasma sample can be incorporated into a DNA sequencing library. Sequencing libraries from several plasma samples can be pooled with control samples, purified, and, in some embodiments, sequenced on Illumina sequencers using a 75-cycle single-end, dual index sequencing kit. Primary sequencing output can be demultiplexed followed by quality trimming of the reads. In some embodiments, the reads that pass quality filters are aligned against human and synthetic references and then excluded from the analysis, or otherwise set aside. Reads potentially representing human satellite DNA can also filtered, e.g., via a k-mer-based method; then the remaining reads can be aligned with a microorganism reference database, (e.g., a database with 20,963 assemblies of high-quality genomic references). In some embodiments, reads with alignments that exhibit both high percent identity and/or high query coverage can be retained, except, e.g., for reads that are aligned with any mitochondrial or plasmid reference sequences. PCR duplicates can be removed based on their alignments. Relative abundances can be assigned to each taxon in a sample based on the sequencing reads and their alignments.

For each combination of read and taxon, a read sequence probability can be defined that accounts for the divergence between the microorganism present in the sample and the reference assemblies in the database. A mixture model can be used to assign a likelihood to the complete collection of sequencing reads that included the read sequence probabilities and the (unobserved) abundances of each taxon in the sample. In some cases, an expectation-maximization algorithm is applied to compute the maximum likelihood estimate of each taxon abundance. From these abundances, the number of reads arising from each taxon can be aggregated up the taxonomic tree. The estimated taxa abundances from the no template control (NTC) samples within the batch can be combined to parameterize a model of read abundance arising from the environment with variations driven by counting noise. Statistical significance values can then be computed for each estimate of taxon abundance in each patient sample. In some embodiments, taxa that exhibit a high significance level, and are one of the 1449 taxa within the reportable range, comprise the candidate calls. Final calls can be made after additional filtering is applied, which accounts for read location uniformity as well as cross-reactivity risk originating from higher abundance calls. The microorganism calls that pass these filters are reported along with abundances in MPM, as estimated using the ratio between the unique reads for the taxon and the number of observed unique reads of normalization molecules.

The amount of mcfDNA plasma concentration in each sample can then be quantified by using the measured relative abundance of the synthetic molecules initially spiked in the plasma.

Analysis

Disclosed herein in some embodiments, are methods of analyzing nucleic acids. Such analytical methods include sequencing the nucleic acids as well as bioinformatic analysis of the sequencing results (e.g., sequence reads).

In some embodiments, a sequencing is performed using a next generation sequencing assay. As used herein, the term “next generation” generally refers to any high-throughput sequencing approach including, but not limited to one or more of the following: massively-parallel signature sequencing, pyrosequencing (e.g., using a Roche 454 GENOME ANALYZER™ sequencing device), ILLUMINA™ (SOLEXA™) sequencing (e.g., using an Illumina NEXTSEQ™ 500), sequencing by synthesis (ILLUMINA™), ion semiconductor sequencing (Ion Torren™), sequencing by ligation (e.g., SOLiD™ sequencing), single molecule real-time (SMRT) sequencing (e.g., PACIFIC BIOSCIENCE™), polony sequencing, DNA nanoball sequencing (COMPLETE GENOMICS™), heliscope single molecule sequencing (Helicos Biosciences™), metagenomic sequencing and nanopore sequencing (e.g., OXFORD NANOPORE™). In some embodiments, a sequencing assay can comprise nanopore sequencing. In some embodiments, a sequencing assay can include some form of Sanger sequencing. In some embodiments, a sequencing can involve shotgun sequencing; in some embodiments, a sequencing can include bridge amplification PCR.

In some embodiments, a sequencing assay comprises a Gilbert's sequencing method. In some embodiments, a Gilbert's sequencing method can comprise chemically modifying nucleic acids (e.g., DNA) and then cleaving them at specific bases. In some embodiments, a sequencing assay can comprise dideoxynucleotide chain termination or Sanger-sequencing.

In some embodiments, a sequencing-by-synthesis approach is used in the methods provided herein. In some embodiments, fluorescently labeled reversible-terminator nucleotides are introduced to clonally-amplified DNA templates immobilized on the surface of a glass flowcell. During each sequencing cycle, a single labeled deoxynucleoside triphosphate (dNTP) may be added to the nucleic acid chain. The labeled terminator nucleotide may be imaged when added in order to identify the base and then the terminator group may be enzymatically cleaved to allow synthesis of the strand to proceed. A terminator group can comprise a 3′-O-blocked reversible terminator or a 3′-unblocked reversible terminator. Since all four reversible terminator-bound dNTPs (A, C, T, G) are generally present as single, separate molecules, natural competition may minimize incorporation bias.

In some embodiments, a method called Single-molecule real-time (SMRT) is used. In such approach, nucleic acids (e.g., DNA) are synthesized in zero-mode waveguides (ZMWs), which are small well-like containers with capturing tools located at the bottom of the well. The sequencing is performed with use of unmodified polymerase (attached to the ZMW bottom) and fluorescently labelled nucleotides flowing freely in the solution. The fluorescent label is detached from the nucleotide upon its incorporation into the DNA strand, leaving an unmodified DNA strand. A detector such as a camera may then be used to detect the light emissions; and the data may be analyzed bioinformatically to obtain sequence information.

In some embodiments, a sequencing by ligation approach is used to sequence the nucleic acids in a sample. One example is the next generation sequencing method of SOLiD (Sequencing by Oligonucleotide Ligation and Detection) sequencing (Life Technologies). This next generation technology may generate hundreds of millions to billions of small sequence reads at one time. The sequencing method may comprise preparing a library of DNA fragments from the sample to be sequenced. In some embodiments, the library is used to prepare clonal bead populations in which only one species of fragment is present on the surface of each bead (e.g., magnetic bead). The fragments attached to the magnetic beads may have a universal P1 adapter sequence attached so that the starting sequence of every fragment is both known and identical. In some embodiments, the method may further involve PCR or emulsion PCR. For example, the emulsion PCR may involve the use of microreactors containing reagents for PCR. The resulting PCR products attached to the beads may then be covalently bound to a glass slide. A sequencing assay such as a SOLiD sequencing assay or other sequencing by ligation assay may include a step involving the use of primers. Primers may hybridize to the P1 adapter sequence or other sequence within the library template. The method may further involve introducing four fluorescently labelled di-base probes that compete for ligation to the sequencing primer. Specificity of the di-base probe may be achieved by interrogating every first and second base in each ligation reaction. Multiple cycles of ligation, detection and cleavage may be performed with the number of cycles determining the eventual read length. In some embodiments, following a series of ligation cycles, the extension product can be removed, and the template can be reset with a primer complementary to the n−1 position for a second round of ligation cycles. Multiple rounds (e.g., 5 rounds) of primer reset may be completed for each sequence tag. Through the primer reset process, each base may be interrogated in two independent ligation reactions by two different primers. For example, a base at read position 5 can be assayed by primer number 2 in ligation cycle 2 and by primer number 3 in ligation cycle 1.

In some embodiments, a detection or quantification analysis of oligonucleotides can be accomplished by sequencing. In some embodiments, entire synthesized oligonucleotides can be detected via full sequencing of all oligonucleotides by e.g., Illumina HiSeq 2500™, including the sequencing methods described herein.

In some embodiments, the sequencing is accomplished through classic Sanger sequencing methods. Sequencing can also be accomplished using high-throughput systems some of which allow detection of a sequenced nucleotide immediately after or upon its incorporation into a growing strand, e.g., detection of sequence in real time or substantially real time. In some embodiments, high throughput sequencing generates at least 1,000, at least 5,000, at least 10,000, at least 20,000, at least 30,000, at least 40,000, at least 50,000, at least 100,000, or at least 500,000 sequence reads per hour. In some embodiments, each read is at least 50, at least 60, at least 70, at least 80, at least 90, at least 100, at least 120, or at least 150 bases per read. In some embodiments, each read is up to 2000, up to 1000, up to 900, up to 800, up to 700, up to 600, up to 500, up to 400, up to 300, up to 200, or up to 100 bases per read. Long read sequencing can include sequencing that provides a contiguous sequence read of longer than 500 bases, longer than 800 bases, longer than 1000 bases, longer than 1500 bases, longer than 2000 bases, longer than 3000 bases, or longer than 4500 bases per read.

In some embodiments, a high-throughput sequencing can involve the use of technology available by Illumina's Genome Analyzer IIX™, MiSeq personal Sequencer™, or HiSeq™ systems, such as those using HiSeq 2500 ™, HiSeq 1500 ™, HiSeq 2000 ™, or HiSeq 1000 ™. These machines use reversible terminator-based sequencing by synthesis chemistry. These machines can sequence 200 billion or more reads in eight days. Smaller systems may be utilized for runs within 3, 2, or 1 days or less time. Short synthesis cycles may be used to minimize the time it takes to obtain sequencing results.

In some embodiments, a high-throughput sequencing involves the use of technology available by ABI Solid System. This genetic analysis platform can enable massively parallel sequencing of clonally amplified DNA fragments linked to beads. The sequencing methodology is based on sequential ligation with dye-labeled oligonucleotides.

In some embodiments, a next-generation sequencing can comprise ion semiconductor sequencing (e.g., using technology from Life Technologies™ (Ion Torrent™)). Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA, an H⁺ ion can be released, which can be measured as a change in pH. The H⁺ ion can be converted to voltage and recorded by the semiconductor sensor. An array chip can be sequentially flooded with one nucleotide after another. In some embodiments, no scanning, light, or cameras are required. In some embodiments, an IONPROTON™ Sequencer is used to sequence nucleic acid. In some embodiments, an IONPGM™ Sequencer is used. The Ion Torrent Personal Genome Machine™ (PGM) can sequence 10 million reads in two hours.

In some embodiments, a high-throughput sequencing involves the use of technology available by Helicos BioSciences Corporation™ (Cambridge, Massachusetts) such as the Single Molecule Sequencing by Synthesis (SMSS) method. SMSS can allow for sequencing the entire human genome in up to 24 hours. In some embodiments, SMSS may not require a pre amplification step prior to hybridization. In some embodiments, SMSS may not require any amplification. In some embodiments, methods of using SMSS are described in part in US Publication Application Nos. 20060024711; 20060024678; 20060012793; 20060012784; and 20050100932, each of which are herein incorporated by reference.

In some embodiments, a high-throughput sequencing involves the use of technology available by 454 Lifesciences, Inc.™ (Branford, Connecticut) such as the Pico Titer Plate™ device which includes a fiber optic plate that transmits chemiluminescent signal generated by the sequencing reaction to be recorded by a charge-coupled device (CCD) camera in the instrument. This use of fiber optics can allow for the detection of a minimum of 20 million base pairs in 4.5 hours. In some embodiments, methods for using bead amplification followed by fiber optics detection are described in Marguiles, M., et al. “Genome sequencing in microfabricated high-density picolitre reactors”, Nature, doi: 10.1038/nature03959; which is herein incorporated by reference.

In some embodiments, high-throughput sequencing is performed using Clonal Single Molecule Array (Solexa, Inc.™) or sequencing-by-synthesis (SBS) utilizing reversible terminator chemistry. Methods of using these technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36, each of which are herein incorporated by reference.

In some embodiments, the next generation sequencing is nanopore sequencing. A nanopore can be a small hole, e.g., on the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies™; e.g., a GridION™ system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiN_{X}, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, a nanopore sequencing technology from GENIA™ can be used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys™. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM™ or Roche™. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics™; see e.g., Drmanac et al. (2010) Science 327: 78-81, which is incorporated herein by reference). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresistant material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

The methods provided herein may include use of a system that contains a nucleic acid sequencer (e.g., DNA sequencer, RNA sequencer) for generating DNA or RNA sequence information. The system may include a computer comprising software or code that performs bioinformatic analysis on the DNA or RNA sequence information. Bioinformatic analysis can include, without limitation, assembling sequence data, detecting and quantifying genetic variants in a sample, including germline variants and somatic cell variants (e.g., a genetic variation associated with cancer or pre-cancerous condition, a genetic variation associated with infection, or a combination thereof). In some embodiments, the bioinformatic analysis determines the threshold value for an assay provided herein, such as a method of determining a response to treatment. In some cases, the bioinformatics analysis further compares the value obtained in a longitudinal sample against the threshold value in order to determine whether there is a response to treatment. In some cases, the threshold value is determined in terms of MPM. In some cases, the bioinformatics analysis applies a known threshold, such as a known threshold value for a particular condition or microbe. For example, in some cases the threshold varies depending on whether an endocarditis patient has a native or prosthetic valve. More specifically, in some embodiments, the threshold value of MPM for the prosthetic valve is higher than that of the native value. In some cases, the bioinformatics analysis uses a program that recognizes and applies different MPM thresholds depending on the condition of the patient (e.g., prosthetic valve, native valve, endocarditis, pneumonia), or the type of microbe.

Sequencing data may be used to determine genetic sequence information, ploidy states, the identity of one or more genetic variants, as well as a quantitative measures of the variants, including relative and absolute relative measures.

In some embodiments a sequencing can involve sequencing of a genome. In some embodiments a genome can be that of a microbe or pathogen as disclosed herein. In some embodiments, sequencing of a genome can involve whole genome sequencing or partial genome sequencing. In some embodiments, a sequencing can be unbiased and can involve sequencing all or substantially all (e.g., greater than 70%, 80%, 90%) of the nucleic acids in a sample. In some embodiments, a sequencing of a genome can be selective, e.g., directed to portions of a genome of interest. In some embodiments, sequencing of select genes, or portions of genes may suffice for a desired analysis. In some embodiments, polynucleotides mapping to specific loci in a genome can be isolated for sequencing by, for example, sequence capture or site-specific amplification.

In some embodiments disclosed herein, is a method comprising a process of analyzing, calculating, quantifying, or a combination thereof. In some embodiments, a method can be used to determine quantities of bacterial and fungal sequence reads. In some embodiments, metrics can be generated to determine quantities of bacterial sequences, fungal sequences, or a combination thereof.

In some embodiments, sensitivity of a test refers to a test's ability to correctly detect subjects with an infection who have an infection. In some embodiments, a sensitivity is a detection rate of a disease or infection. In some embodiments, a sensitivity is the proportion of people who test positive for a disease among those who have the disease. In some embodiments, a sensitivity can be calculated using the following formula: Sensitivity=(number of true positives)/(number of true positives+number of false negatives) or Sensitivity=(number of true positives)/(total number of sick individuals in a population); or Sensitivity=probability of a true positive.

In some embodiments, a specificity can refer to a test's ability to correctly reject healthy subjects without an infection. In some embodiments, a specificity of a test can comprise a proportion of subjects who truly do not have an infection who test negative for the infection. In some embodiments, a specificity can be calculated using the following formula: Specificity=(number of true negatives)/(number of true negatives+number of false positives) or Specificity=(number of true negatives)/(total number of well individuals in a population); or Specificity=probability of a negative test when the patient is healthy or well. In some cases, specificity is the proportion of negative control samples for which no bacterial or fungal organisms were identified by mcfDNA sequencing.

In some embodiments, the quantity for each organism identified in a method provided herein is expressed in Molecules Per Microliter (MPM), the number of DNA sequencing reads from the reported organism present per microliter of plasma. In some cases, detection or prediction of infection (or prediction of onset of symptoms of infection) occurs when the MPM is greater than a threshold value. In some cases, such threshold value of MPM may be greater than 10, 15, 20, 30, 50, 100, 200, 300, 400, 500, 600, 700, 800, 900, 1000, 2000, 5000, 7000, 10000, 20000, 30000, or 40000. In some cases, the MPM threshold is determined for a particular organism. For example, in some embodiments, the MPM threshold for detection of native valve endocarditis is 2000, 3881, 4000, 6000, 8000, 10000, 12000, 15000, 17000, or 20000. In some embodiments, the MPM threshold for detection of prosthetic valve endocarditis is 40000, 47273, 50000, 60000, 70000, 80000 or 100000. In some embodiments, the MPM threshold for detection of endocarditis generally is 30000, 38610, 40000, 47273, 50000, 60000, 70000, 80000 or 100000, or any range within these values. In some cases, the method applies a MPM threshold for prosthetic valve endocarditis that is higher than the MPM threshold for native valve endocarditis.

In some embodiments, the quantity for a microbe (e.g., bacterium, fungus, virus) identified in a method provided herein is expressed as the amount or quantity of the microbe in a sample in relation to, or compared with, a threshold value, e.g., the amount of microbial cell-free nucleic acid in a sample as a percentage of the amount of the microbial cell-free nucleic acid in an initial sample. In some cases, the threshold value is an absolute value that can be used generally, irrespective of the subject. For example, the threshold value may be a normalized value signifying an average MPM value for a particular microbe in samples from a cohort of infected individuals prior to starting treatment for the infection. In some embodiments, the threshold value is the amount of a microbe measured in the initial sample (e.g., plasma, serum, cell-free sample) that is collected from the patient before beginning the treatment regimen for the microbial infection or while the patient is undergoing the treatment regimen for the microbial infection (e.g., in the initial stages of undergoing such treatment regimen). Preferably, the amount of a microbe is based on measurements of microbial cell-free nucleic acid (mcfNA). Preferably the mcfNA is microbial cell-free DNA (mcfDNA). The amount of mcfNA may be expressed in MPM. In some cases, the MPM is an adjusted or normalized value. For example, the MPM may be adjusted based on the quantity of synthetic nucleic acids detected.

As used herein, a sample collected after an initial sample is a “longitudinal sample” or “longitudinal plasma sample.” In some cases, the MPM threshold is determined for a particular microbe, preferably the microbe is the microbe associated with the microbial infection of the patient. In some cases, the amount of the mcfNA (e.g., MPM) compared to a threshold value may indicate a subject's response to a treatment. In some cases, a response to treatment is indicated when the amount of mcfNA in the longitudinal plasma sample is 10%-100% lower than the threshold value, or the amount of mcfNA in the longitudinal plasma sample is 25%-100% lower than the threshold value, or the amount of mcfNA in the longitudinal plasma sample is 50%-100% lower than the threshold value, or the amount of mcfNA in the longitudinal plasma sample is 75%-100% lower than the threshold value. In some cases, a response to treatment is indicated when the amount of mcfNA in the longitudinal plasma sample is at least about 10%, 20%, 25%, 30%, 35%, 40%, 45%, 50%, 55%, 70%, 80%, 90% or 100% lower than the threshold value. By way of example, if the threshold value is 100 MPM, an MPM of 25 for a longitudinal sample indicates a value that is 75% lower than the threshold value.

Treating

In some embodiments, the methods provided herein comprise administering a treatment to a subject. In some cases, the methods comprise following a subject over time in order to monitor the subject's response to a treatment. In some cases, a sample is taken from the subject at one point in time after administration of a treatment in order to determine a subject's response to treatment. In some cases, the subject is on a treatment regimen. In some embodiments, the treatment regimen is a plan that sets forth the dosage, schedule, and/or duration of treatment, in any combination.

In some embodiments, the treatment is an antimicrobial treatment. The antimicrobial treatment can, in some instances, be administered to the subject when the subject is culture positive for the infection, e.g., blood culture positive or biopsy or tissue culture positive. In some embodiments, the treatment is administered to a subject when the subject is blood culture negative for the microbe that is the target of the treatment. In some embodiments, the treatment treats or reduces symptoms of an infection.

Various non-limiting treatments can be administered to the subject. Likewise, the methods provided herein can involve monitoring a subject's response to various treatments. In some embodiments, the treatment is a broad-spectrum antimicrobial drug or an antimicrobial drug that targets a specific microbe or a specific class of microbes. In some embodiments, the treatment targets bacteria and/or fungi. In some embodiments, the subject is treated, or has been treated, with a combination of drugs (e.g., a combination of multiple antibiotics, multiple anti-fungal drugs, or both antibiotics and antifungal drugs). In some embodiments, the subject is treated, or has been treated, with a combination of broad-spectrum antibiotics, a combination of broad- and narrow-spectrum antibiotics, a combination of narrow-spectrum antibiotics, a combination of broad-spectrum antifungals, a combination of broad and narrow-spectrum antifungals, or a combination of narrow-spectrum antifungals. In some embodiments, the subject is treated, or has been treated, with a broad-spectrum antibiotic, a narrow-spectrum antibiotic, a broad-spectrum antifungal, a narrow-spectrum antifungal, or any combination thereof.

In some embodiments, the treatment is an antimicrobial. In some embodiments, an antimicrobial comprises a β-lactam, an aminoglycoside, a quinolone, an oxazolidinone, a sulfonamide, a macrolide, a tetracycline, an ansamycin, a streptogramin, a lipopeptide, used singly, or in any combination thereof as used herein and/or as recommended by a clinician. In some embodiments, the treatment is a broad-spectrum treatment. In some embodiments, the broad-spectrum treatment is a broad-spectrum antibiotic, a broad-spectrum anti-bacterial drug, a broad-spectrum antifungal, or any combination thereof. As used herein, the term “broad spectrum antibiotic” generally refers to a drug that acts on both gram negative and gram-positive bacteria, that acts on multiple types of gram-negative bacteria, and/or that acts on multiple types of gram-positive bacteria. In some embodiments, the broad-spectrum treatment acts on multiple types of fungal infections. In some embodiments, the broad-spectrum drug is a broad-spectrum non-limiting examples include β-lactam penicillin such as flucloxacillin, ampicillin (or amoxicillin). In some embodiments, the broad-spectrum drug is a β-lactam such as cephalosporin antibiotic (e.g., ceftriaxone, cefepime). The cephalosporin drug can be, in some embodiments, a first, second, third or fourth generation cephalosporin drug. In some embodiments, the broad-spectrum antibiotic is a quinolone drug (e.g., levofloxacin), a carbopenem-type antibiotic (e.g., meropenem), or a metronidazole.

In some embodiments, the broad-spectrum treatment is an antifungal drug. In some embodiments, the antifungal drug is, for example, a cefepime, a clotrimazole, a econazole, a miconazole, a terbinafine, a fluconazole, a ketoconazole, a nystatin, an amphotericin B, or any other known antifungal drugs and/or a combination thereof.

In some embodiments, the treatment is a narrow-spectrum antimicrobial drug. In some embodiments, the narrow-spectrum antimicrobial drug is a vancomycin, a glycopeptidic antibiotic active against gram-positive bacteria. In some embodiments, the narrow-spectrum drug can comprise various narrow-spectrum drugs, for example, a flucytosine. In some embodiments, the narrow-spectrum drug can comprise an oxazolidinone, for example, a linezolid, a posizolid, a radezolid, a penicillin VK, or any combination thereof.

In some embodiments, the antimicrobial drug is a pill, a gel, a tablet, a coated tablet, or any combination thereof and can be administered to the subject orally. In some embodiments, the treatment using an anti-fungal can be administered to the subject topically. In some embodiments, a topical administration can comprise administering the treatment as a cream, a gel, an ointment, a spray, or any combination thereof. In some embodiments, a treatment can be administered in the form of a capsule, a tablet, a liquid, an injectable, a pessary or any combination thereof. In some embodiments, the antimicrobial drug is formulated as an infusion, and can be administered to the subject intravenously via a needle or catheter.

In some embodiments, the methods comprise a method of monitoring a response to treatment or response to a treatment regimen. In some cases, at least one sample is collected from the subject prior to starting treatment regimen or while on a treatment regimen. Such sample may be an initial sample. In some cases, the initial sample is spiked with synthetic nucleic acids (e.g., a known quantity of nucleic acids). In some cases, the method comprises detecting microbial cell-free nucleic acids (e.g., mcfDNA) in the initial sample. The method can further comprise quantifying the mcfNA (e.g., mcfDNA). The quantifying can be done in some cases, by comparing the detected quantity of mcfNA against a detected quantity of a known quantity of synthetic nucleic acids (sNA). In some cases, the amount of mcfNA (e.g., mcfNA from a particular bacterium, fungus, or virus) is used to obtain a threshold value. In some cases, the method further comprises obtaining a longitudinal sample at a later timepoint from the same subject. In some cases, the longitudinal sample is spiked with sNA. In some cases, the method comprises detecting an amount of mcfNA (e.g., mcfDNA) in the longitudinal sample. In some cases, the amount of mcfNA is determined based on the detected quantity of a known quantity of sNA. The method can further comprise comparing the mcfNA in the longitudinal sample with the threshold value in order to determine whether the patient or subject has responded to the treatment. In some cases, a decrease in mcfNA compared with the threshold value is indicative that the patient is responding to treatment at the time the longitudinal sample is collected. In some cases, the comparison of the longitudinal mcfNA to the threshold value is provided as a percentage, as described herein. In some cases, the comparison is expressed as a ratio.

### EXAMPLES

### Example 1—Bacteria

Bartonella henselae and Bartonella quintana are the etiologic agents of cat scratch disease and “trench fever,” respectively. Both are important causes of culture-negative endocarditis.

There are several hindrances to the diagnosis of Bartonella infections: (1) the fastidious nature of Bartonella spp., leads to rare detections with traditional culture-based methods and a lack of reliable, widely available diagnostic tests; (2) the nonspecific manifestations of the disease; and (3) failure to obtain a history of exposure risk factors for Bartonella infection.

Here results of diagnosis of 23 Bartonella infections from August 2017 are disclosed to present using next-generation sequencing (NGS) of microbial cell free DNA (mcfDNA).

The method of detection was an NGS test that detected mcfDNA in plasma. After mcfDNA was extracted and NGS performed, human reads were removed, and remaining sequences were aligned to a curated database of >1400 organisms. McfDNA from organisms present above a statistical threshold were reported and quantified in MPM. Case review was performed by infectious disease experts.

In total, ten cases of endocarditis, twelve cases of cat scratch disease/fever of unknown origin (CSD/FUO), and a single case of osteomyelitis were analyzed (Table 1). The method disclosed herein detected Bartonella henselae mcfDNA in 22 cases and detected Bartonella quintana mcfDNA in one pediatric case of prosthetic valve endocarditis. Only one patient in this cohort was immunocompromised. Glomerulonephritis was reported in four of the endocarditis patients. Two cases had hepatic involvement and six had splenic involvement. A history of exposure to cats was elicited in seven of the cases.

The mean MPMs was highest for prosthetic valve endocarditis (mean 47,272+/−67,526) followed by native valve endocarditis (3,881+/−2,458), FUO/CSD (1,922+/−3,416), and osteomyelitis (119+/−0) (p<0.05).

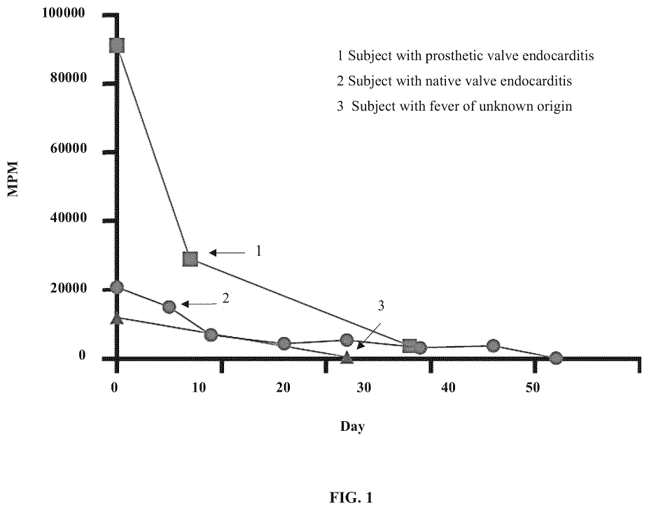

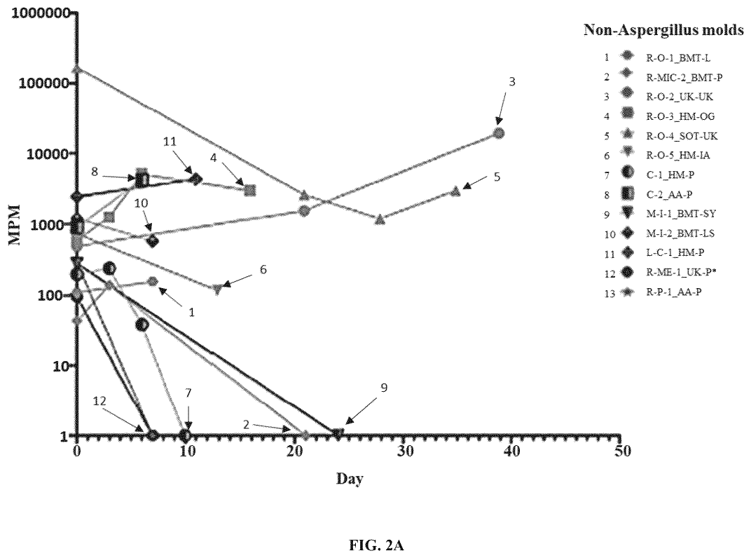

Serial longitudinal samples were obtained in three cases (FIG. 1). In the first, a native valve endocarditis case, eight samples were sent over a six-week period with a decline in MPMs from 20,804 on the first KT sent down to 89 MPMs six weeks later. In the second, a prosthetic valve endocarditis case, three samples were sent over a four-week period with a decline in MPMs from 91,221 in the first sample down to 3,708 MPMs 28 days later. In the third case, an immunocompromised patient with fever of unknown origin, a decline in MPMs was observed from 11,926 in the first sample down to 489 MPMs 22 days later. FIG. 1 shows the results of serial test sampling of a Bartonella infection. Molecules per microliter (MPM) was measured over several days after a threshold test. Data was collected from three patients: (e) one with native valve endocarditis; (n) one with prosthetic valve endocarditis; and (A) one with fever of unknown origin.

The disclosure herein demonstrates that open-ended, plasma based NGS for mcfDNA provides a rapid, non-invasive method to diagnose pediatric cases of Bartonella spp. infection. Furthermore, these cases highlight the potential of this technique to diagnose infections caused by fastidious/unculturable pathogens. Additionally, the level of MPMs may help in differentiating disease caused by Bartonella. Finally, serial monitoring to trend MPMs may provide a way to monitor appropriate treatment response.

### Example 2—Fungi

Diagnostic results were reviewed for detections of Aspergillus, non-Aspergillus molds and Pneumocystis jirovecii (PJ) in children. The methods disclosed herein used to detect microbial cell-free DNA (mcfDNA) can assist with the diagnosis of invasive infections. McfDNA was extracted, NGS was performed, human sequences were removed, and remaining sequences were aligned to a curated pathogen database of >1400 organisms. Organisms present above a statistical threshold were reported and quantified. For >85% of tests the time to result reporting was 24 hours from sample receipt. Clinical information was included from data submitted with the requisition or obtained at the time of reporting from clinical consultations with the provider.

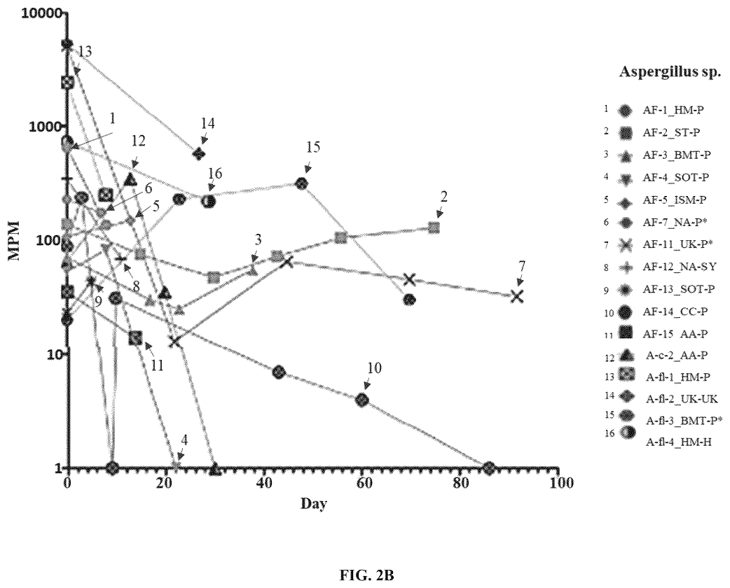

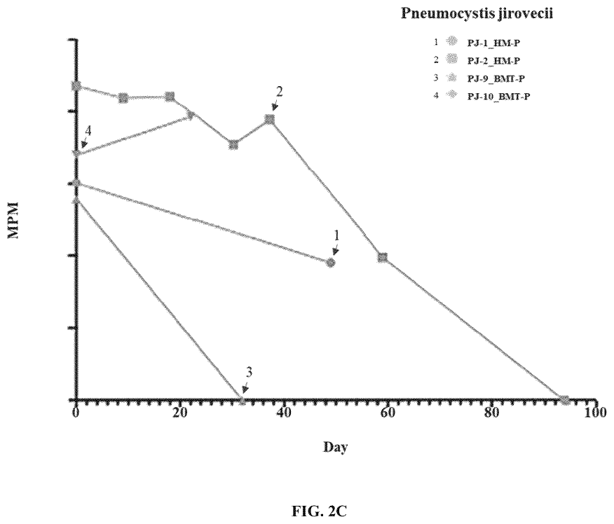

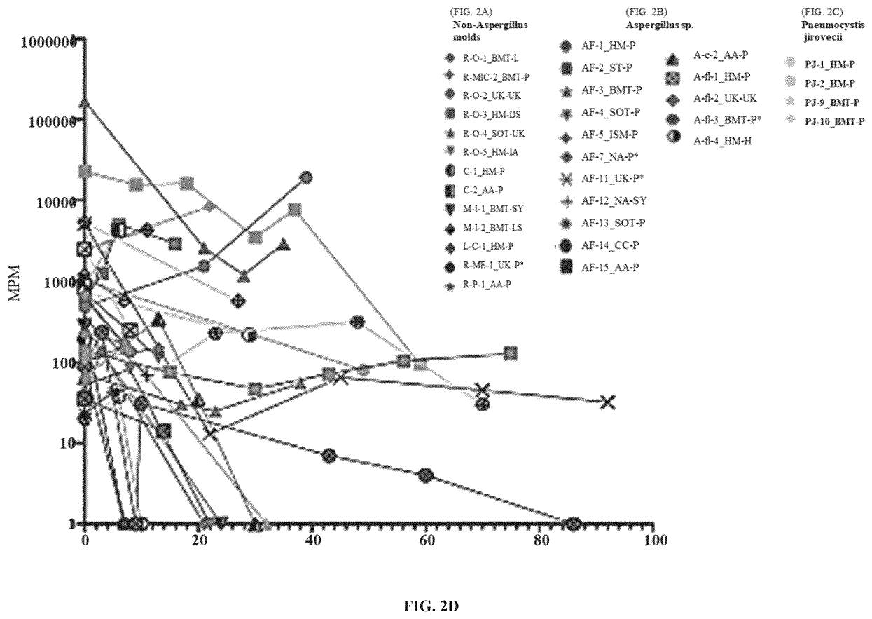

Seven different species of Aspergillus were detected in 61 patients (74% IC, 40% with a pulmonary focus). Fifteen different non-Aspergillus molds were detected in 51 patients (80% IC, 36% with a pulmonary focus). Pneumocystis jirovecii was detected in 37 patients (73% IC, 76% with a pulmonary focus, 54% with a DNA virus co-detection and 32% with a herpesvirus co-detections). There were 31 subjects with serial monitoring (97% IC, 70% with a pulmonary focus) including 48% with Aspergillus, 39% with non-Aspergillus molds and 12% with PJ. 71% of subjects demonstrated a decline in the quantitative mcfDNA signal over time; the duration of a positive mcfDNA signal ranged from 3-92 days (median 16 days, SD 22.4). FIG. 2 shows the results of serial test sampling of a fungal infection. Molecules per microliter (MPM) was measured over several days after a threshold test. FIG. 2A shows results from patients infected with non-Aspergillus sp. molds. The following codes were used to identify individuals: R-O-1_BMT-L; R-MIC-2_BMT-P; R-O-2_UK-UK; R-O-3_HM-OG; R-O-4_SOT-UK; R-O-5_HM-IA; C-1_HM-P; C-2_AA-P; M-I-1_BMT-SY; M-I-2_BMT-LS; L-C-1_HM-P; R-ME-1_UK-P*; R-P-1_AA-P. FIG. 2B shows results from patients infected with Aspergillus. The following codes were used to identify individuals: AF-1 HM-P; AF-2_ST-P; AF-3_BMT-P; AF-4_SOT-P; AF-5_ISM-P; AF-7_NA-P*; AF-11_UK-P*; AF-12_NA-SY; AF-13_SOT-P; AF-14_CC-P; AF-15_AA-P; A-c-2_AA-P; A-fl-1_HM-P; A-fl-2_UK-UK; A-fl-3_BMT-P*; A-fl-4_HM-H. FIG. 2C shows results from patients infected with PJ. The following codes were used to identify individuals: PJ-1_HM-P; PJ-2 HM-P; PJ-9_BMT-P, PJ-10_BMT-P. FIG. 2D shows a composite of FIG. 2A, FIG. 2B, and FIG. 2C The following coding was used in FIG. 2A, FIG. 2B, FIG. 2C and FIG. 2D: Genus/Species: AF—Aspergillus fumigatus; A-c—Aspergillus calidoustus; A-fl—Aspergillus flavus oryzae; R-D—Rhizopus delemar; R-MIC—Rhizopus microsporus; R-O—Rhizopus oryzae; C—Cunninghamella; M-I—Mucor indicus; L-C—Lichtheimia corymbifera; R-MIE—Rhizomucor miehei; R-P—Rhizopus pusillus; PJ—Pneumocystis jirovecii. Underlying conditions: HM—Hematologic Malignancy; SOT—Solid Organ Transplant; BMT—Bone Marrow Transplant; ST—Solid Tumor; ISM—Immunosuppressing Medications; CC—Cardiac Congenital Disease; AA—Aplastic Anemia; UK—Unknown; NA—None. Focus on indications: P—Pulmonary; SY—Systemic; H—Heart; LS—Liver/Spleen; IA—Intra-abdominal; UK—Unknown.

The disclosure herein shows that plasma mcfDNA NGS offers a rapid, non-invasive means of detecting a broad diversity of invasive pathogens that overlap in their clinical presentations and are difficult to identify in immunocompromised children. The rapid turnaround time, non-invasive sampling, and 1-sample-1000+test-solution may lead to a faster time to pathogen diagnosis, faster time to targeted therapy and obviate the need for invasive diagnostic procedures. The ability with a single test to concomitantly diagnose co-pathogens including reactivating herpesviruses that modulate the progression of principal infecting fungal pathogens (i.e., cytomegalovirus modulation of PJ) can help optimize care. Additionally, this convenient non-invasive means of serial testing of invasive fungal infections may serve as an indicator of burden of infection, provide insight into treatment efficacy and ultimately help define the length and mode (medical/surgical) of therapy required to improve outcomes. Additional studies correlating the mcfDNA signal with individual patient clinical and radiographic parameters will be important to further define the utility of serial mcfDNA monitoring.

TABLE 1
Pediatric Bartonella Cases

| | | Bartonella | | | | | | |
|---|---|---|---|---|---|---|---|---|
| Clinical | | mcfDNA | Immunocom- | | | | Lymph- | Cat |
| Manifestation | n/% | MPM Mean/SD | promised | Renal | Hepatic | Splenic | adenitis | Exposure |
| Total | 23/100% | 20,269/ | 1 | 5 | 3 | 6 | 3 | 8 |
| | | 41,693 | | | | | | |
| Endocarditis | 10/43.5% | 38,610/ | 0 | 5 | 0 | 3 | 0 | 2 |
| Total | | 52,702 | | | | | | |
| Native valve | 3/13% | 3,881/ | 0 | 2 | 0 | 1 | 0 | 1 |
| | | 2,458 | | | | | | |
| Prosthetic | 7/30.4% | 47,273/ | 0 | 3 | 0 | 2 | 0 | 1 |
| valve | | 67,426 | | | | | | |
| Fever of | 12/52.2% | 1,922/ | 1 | 0 | 2 | 2 | 3 | 5 |
| unknown | | 3,416 | | | | | | |
| origin/Cat | | | | | | | | |
| Scratch | | | | | | | | |
| Disease | | | | | | | | |
| Osteomyelitis | 1/4.3% | 119/— | 0 | 0 | 1 | 1 | 0 | 1 |

All cases were Bartonella henselae except one patient with prosthetic valve endocarditis in which Bartonella quintana was found.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-73. (canceled)

74. A method of detecting an alteration of a prosthetic valve, wherein the method comprises:
a. providing a plasma sample from a subject suspected of having an alteration of a prosthetic valve, wherein the alteration of the prosthetic valve is prosthetic valve endocarditis;
b. performing massively parallel sequencing on cell-free DNA from the plasma sample to obtain cell-free DNA sequence reads comprising cell-free DNA sequence reads derived from Bartonella bacteria associated with the prosthetic valve endocarditis;
c. detecting an amount of the cell-free DNA sequence reads derived from Bartonella bacteria associated with the prosthetic valve endocarditis; and
d. identifying the prosthetic valve endocarditis in the subject when the amount of the cell-free DNA sequence reads derived from Bartonella bacteria associated with the prosthetic valve endocarditis exceeds a threshold level.

75. The method of claim 74, wherein the Bartonella bacteria associated with the prosthetic valve endocarditis is Bartonella henselae or Bartonella quintana.

76. The method of claim 74, wherein the Bartonella bacteria associated with the prosthetic valve endocarditis is Bartonella henselae.

77. The method of claim 74, wherein the amount of cell-free DNA sequence reads derived from the Bartonella bacteria associated with the prosthetic valve endocarditis is quantified as molecules per microliter (MPM), and wherein the threshold level is greater than 100 MPM.

78. The method of claim 74, wherein the amount of cell-free DNA sequence reads derived from the Bartonella bacteria associated with the prosthetic valve endocarditis is quantified as molecules per microliter (MPM), and wherein the threshold level is greater than 10,000 MPM.

79. The method of claim 74, wherein the subject suspected of having an alteration of a prosthetic valve has cat-scratch fever or trench fever.

80. The method of claim 74, wherein the subject suspected of having an alteration of a prosthetic valve has a prosthetic valve selected from the group consisting of a partial heart valve and a complete heart valve.

81. The method of claim 74, wherein the subject suspected of having an alteration of a prosthetic valve has a glomerulonephritis.

82. The method of claim 74, wherein the subject suspected of having an alteration of a prosthetic valve is immunocompromised.

83. The method of claim 74, wherein the subject suspected of having an alteration of a prosthetic valve received an immunosuppressant.

84. The method of claim 74, wherein the subject suspected of having an alteration of a prosthetic valve has a negative blood culture.

85. The method of claim 74, wherein the subject suspected of having an alteration of a prosthetic valve has a fever.

86. The method of claim 74, wherein the method further comprises a method of treatment comprising administering a treatment regimen to the subject suspected of having an alteration of a prosthetic valve to treat the prosthetic valve endocarditis.

87. The method of claim 86, wherein the treatment regimen comprises administering at least one drug selected from the group consisting of a penicillin, tetracycline, vancomycin, cefepime, meropenem, doxycycline, cephalosporin, and aminoglycoside.

88. The method of claim 86, wherein the treatment regimen comprises a prosthetic valve replacement.

89. A method of treating endocarditis or osteomyelitis, wherein the method comprises:
a. providing a plasma sample from a subject suspected of having endocarditis or osteomyelitis;
b. performing massively parallel sequencing on cell-free DNA from the plasma sample to obtain cell-free DNA sequence reads comprising cell-free DNA sequence reads derived from Bartonella bacteria associated with endocarditis or osteomyelitis;
c. detecting an amount of the cell-free DNA sequence reads derived from Bartonella bacteria associated with the endocarditis or osteomyelitis;
d. identifying the endocarditis or osteomyelitis in the subject when the amount of the cell-free DNA sequence reads derived from Bartonella bacteria associated with the endocarditis or osteomyelitis exceeds a threshold level; and
e. administering a treatment regimen to the subject suspected of having endocarditis or osteomyelitis to treat the endocarditis or osteomyelitis.

90. The method of claim 89, wherein the endocarditis is native valve endocarditis.
